(19) United States
(12) Patent Application Publication
(10) Pub. No.: US 2024/0408127 A1
(43) Pub. Date: Dec. 12, 2024

(54) SHORT AMORPHOUS CELLULOSE POLYMERS

(71) Applicant: Katholieke Universiteit Leuven, Leuven (BE)

(72) Inventors: Christophe COURTIN, Wilsele (BE); Bert SELS, Beverlee (BE); Karel THIELEMANS, Rotselaar (BE); Sander VAN DEN BOSCH, Boortmeerbeek (BE); Aron DENEYER, Beverlee (BE); Chiara ROYE, Beverlee (BE)

(21) Appl. No.: 18/699,983

(22) PCT Filed: Oct. 12, 2022
(86) PCT No.: PCT/EP2022/078440

§ 371 Date: Apr. 10, 2024

(30) Foreign Application Priority Data
Oct. 12, 2021 (EP) 21202228.9

(51) Int. Cl. A61K 31/717; A21D 2/18; A23L 33/24; C08B 15/02

(52) U.S. Cl. CPC A61K 31/717; A21D 2/188; A23L 33/24; C08B 15/02

(57) ABSTRACT

The invention relates to the use of a cellulose with an average degree of Polymerization (DP) below 100 and a crystallinity index below 0.5 as a fermentable dietary fiber in a food composition.

### FIELD OF THE INVENTION

The invention relates to the processing of cellulose material and its use as a digestible dietary fiber and as treatment for colon disorders.

### BACKGROUND OF THE INVENTION

Cellulose, a β-1,4-bond glucose polymer, is the most abundant renewable material in nature, being the main building block of the plant cell wall. Here, linear glucose polymers are closely packed with strong hydrogen interactions between the different polymers, resulting in long rigid (crystalline) cellulose fibres [Chen (2005), Biotechnology of Lignocellulose]. Today, cellulose is of growing interest for high-end valorisation: they can be used as glucose feedstock in the biorefinery or as bio-based fiber for its distinct mechanical properties [Cherubini (2010) Energy Conversion and Management 51 (7), 1412-1421; Bozell and Petersen (2010) Green Chemistry 12 (4), 5392]. Refined cellulose fibres, such as microcrystalline cellulose or nanocelluloses, are purified and partially hydrolysed, and are widely used in, e.g., packaging material, coatings, membranes, analytics, construction material, drug delivery and/or in the food industry. The crystallinity of these fibres (mostly obtained after homogenization) should be high enough to ensure the strength of the fiber compositions [Brinchi et al. (2013) Carbohydrate polymers 94 (1), 154-169; An et al. (2016), Cellulose 23 (4), 2409-2420], and therefore, at the moment, decreasing the crystallinity of microcrystalline cellulose or nanocellulose is not targeted. However, it is our strong belief that decreasing the crystallinity of the refined fibres can be relevant for unexploited valorisations, where high accessibility of the fibres would be advantageous. The crystallinity of the cellulose fibres (partially hydrolysed or native) is nevertheless one of the main reasons cellulose is difficult to degrade [Xu et al. (2012) Biotechnology for Biofuels 5 (1), 58].

In the human gut, for example, cellulose is not fermented in the large intestine, as colon microbiota cannot access the β-1,4-bonds of the glucose-polymers [Cummings et al. (1984) Gut 25 (8), 805-810]. However, fermentation of dietary fibres like cellulose in the colon is correlated with different positive health effects, linked to the production of short-chain fatty acids (SCFA), which provides the colon cells with energy and contribute to a balanced glucose and cholesterol metabolism [den Besten et al. (2013) J. lipid res. 54 (9), 2325-2340]. In addition, fermentation of carbohydrates causes a decrease in the pH in the lumen and repression of pathogen growth [Wong et al. (2006) J. Clin. Gastroenterology 40 (3), 235-243].

Since the average daily intake of dietary fiber is (still) too low in current Western diets [“Scientific Opinion on Dietary Reference Values for Carbohydrates and Dietary Fibre” (2010) EFSA Journal 8 (3), 1462], the food industry is continuously searching for valuable dietary fiber components. Existing fiber additives (e.g. wheat bran) are often linked with organoleptic and/or techno-functional shortcomings [Hemdane et al. (2015) Food Chemistry 187, 280-289], while new introductions (e.g. arabinoxylan-oligosaccharides from wheat bran) have been repressed by too high production costs [Swennen et al. (2006) J. Sci. Food and Agriculture 86 (11), 1722-1731]. On the other hand, introductions with inulin, or inulin-derived oligosaccharides raise questions about sustainable production, and are fermented too fast, causing flatulencies, diarrhoea and other discomforts, especially for patients with inflammatory bowel disease (IBD) [Simões et al. (2022). European journal of nutrition, 1-12]. The search for new fiber additives is, therefore, still very relevant. The exploitation of cellulose for this purpose will have a large impact due to the abundant character of the material. Cellulose is already used today as a low-fat substituent, bulking agent, thickener and/or emulsion-stabilizer [Mirhosseini et al., (2008) Carbohydrate Polymers 73 (1), 83-91], but the physiological health benefits of cellulose are limited to an increased faecal bulk, due to the recalcitrance of the fibres.

U.S. Pat. No. 4,357,467 discloses depolymerised cellulosic material with low crystallinity.

### SUMMARY

The present invention provides a food-grade modification strategy of cellulose towards higher overall accessibility in order to increase the fermentability of cellulose in the colon.

A carbohydrate fiber composition containing accessible cellulose structures is generated by the combination of mechanical impact and chemical hydrolysis with organic acids. A subsequent mechanical post-treatment of these short cellulose fibres after hydrolysis improves the physiological effects of the fiber composition for use in the food industry, as their fermentability in the colon is increased. In vitro fermentation experiments with the cellulose obtained after this triple treatment showed that the pH decreased, protein fermentation was inhibited, and the proportion of butyrate production was enhanced, compared to cellulose which underwent a single mechanical treatment and hydrolysis.

The present invention allows decreasing the recalcitrance of cellulose (such as Avicel) in a food grade way, without losing the insoluble character of the fibres. In a particular embodiment, a planetary ball mill treatment, chemical hydrolysis with citric acid, and an additional ball mill treatment resulted in fibres with a crystallinity index below 0.5 and an average DP between 20 AGU and 50 AGU. The recalcitrance of these short amorphous cellulose (SAC) fibres was decreased since these fibres were proven to be more accessible for enzymatic blends than unmodified cellulose (<0.4 conversion factor after 1 h hydrolysis with CTec2 enzyme blend).

This enhanced accessibility results in enhanced health benefits when the cellulose is ingested, thanks to an enhanced fermentation in the large intestine. The minimal fermentation degree of 7.6% for prior art cellulose could be enhanced towards at least 45.8%, based on an in vitro fermentation experiment. Furthermore, the main cellulose fermentation happened after 8 h of incubation, which will limit the discomforts of carbohydrate fermentation for suspects with IBD.

The invention is summarised in the following statements:
1. Use of a cellulose with an average degree of Polymerization (DP) below 100 and a crystallinity index below 0.75 as a fermentable dietary fiber in a food composition,
wherein DP is determined viscometrically, and
wherein crystallinity index is determined by X-ray diffraction.
2. The use according to statement 1, wherein the food composition is a cereal product.
3. The use according to statement 1 or 2, wherein the food composition is a bread.
4. The use according to any one of statements 1 to 3, as a fermentable dietary fiber producing acetate, butyrate and propionate.
5. The use according to any one of statements 1 to 4, wherein the cellulose has a fermentation degree between 7.6 and 45.8% upon fermentation.
6. The use according to any one of statements 1 to 5, wherein the cellulose has an average degree of Polymerization (DP) below 100 and a crystallinity index below 0.5.
7. The use according to any one of statements 1 to 6, wherein the crystallinity index of said cellulose is below 0.45.
8. The use according to any one of statements 1 to 7, wherein the crystallinity index of said cellulose is below 0.40.
9. The use according to any one of statements 1 to 8, wherein the crystallinity index of said cellulose is below 0.35.
10. The use according to any one of statements 1 to 9, wherein the crystallinity index of said cellulose is below 0.33.
11. The use according to any one of statements 1 to 10, wherein the average degree of polymerization of said cellulose is below 80.
12. The use according to any one of statements 1 to 11, wherein the average degree of polymerization of said cellulose is below 60.
13. The use according to any one of statements 1 to 12, wherein the average degree of polymerization of said cellulose is below 50.
14. The use according to any one of statements 1 to 13, wherein the average degree of polymerization of said cellulose is below 40.
15. The use according to any one of statements 1 to 14, wherein the average degree of polymerization of said cellulose is below 30.
Uses with all combinations of above-mentioned DP values and above mention CI values are explicitly disclosed and envisaged.
16. The use according to any one of statements 1 to 15, wherein the crystallinity index of said cellulose is below 0.33 and the average degree of polymerization is below 50.
17. A bakery product comprising cellulose with an average degree of Polymerization (DP) below 100 and a crystallinity index below 0.75,
wherein DP is determined viscometrically, and
wherein crystallinity index is determined by X-ray diffraction.
18. The bakery product according to statement 17, which is a bread.
19. The bakery product according to statement 17, which is a wholemeal bread.
20. The bakery product according to any one of statements 17 to 19, comprising between 5-30, 5-25, 5-20, 5-15, 5-10, 10-30, 10-25, 10-20, 10-15 wt % of said cellulose.
21. The bakery product according to any one of statements 17 to 20, wherein the cellulose has a fermentation degree between 7.6 and 45.8%.
22. The bakery product according to any one of statements 17 to 21, wherein the cellulose has an average degree of Polymerization (DP) below 100 and a crystallinity index below 0.5.
23. The bakery product according to any one of statements 17 to 22, wherein the crystallinity index of said cellulose is below 0.45.
24. The bakery product according to any one of statements 17 to 23, wherein the crystallinity index of said cellulose is below 0.40.
25. The bakery product according to any one of statements 17 to 24, wherein the crystallinity index of said cellulose is below 0.35.
26. The bakery product according to any one of statements 17 to 25, wherein the crystallinity index of said cellulose is below 0.33.
27. The bakery product according to any one of statements 17 to 26, wherein the average degree of polymerization of said cellulose is below 80.
28. The bakery product according to any one of statements 17 to 27, wherein the average degree of polymerization of said cellulose is below 60.
29. The bakery product according to any one of statements 17 to 28, wherein the average degree of polymerization of said cellulose is below 50.
30. The bakery product according to any one of statements 17 to 29, wherein the average degree of polymerization of said cellulose is below 40.
31. The bakery product according to any one of statements 17 to 30, wherein the average degree of polymerization of said cellulose is below 30.
Bakery products with all combinations of above-mentioned DP values and above mention CI values are explicitly disclosed and envisaged.
32. The bakery product according to any one of statements 17 to 31, wherein the crystallinity index of said cellulose is below 0.33 and the average degree of polymerization is below 50.
33. Cellulose with an average degree of Polymerization (DP) below 100 and a crystallinity index below 0.75,
wherein DP is determined viscometrically, and
wherein crystallinity index is determined by X-ray diffraction, for use in the treatment of colon disorders.
34. Cellulose with an average degree of Polymerization (DP) below 100 and a crystallinity index below 0.75 as, for use according to statement 33, in the treatment inflammation in colon disorders.
35. Cellulose for use in the treatment according to statement 33 or 34, wherein the cellulose has a fermentation degree between 7.6 and 45.8%.
36. Cellulose for use in the treatment according to any one of statements 33 to 35, wherein the cellulose has an average degree of Polymerization (DP) below 100 and a crystallinity index below 0.5.
37. Cellulose for use in the treatment according to any one of statements 33 to 36, wherein the crystallinity index of said cellulose is below 0.45.
38. Cellulose for use in the treatment according to any one of statements 33 to 37, wherein the crystallinity index of said cellulose is below 0.40.
39. Cellulose for use in the treatment according to any one of statements 33 to 38, wherein the crystallinity index of said cellulose is below 0.35.
40. Cellulose for use in the treatment according to any one of statements 33 to 39, wherein the crystallinity index of said cellulose is below 0.33.
41. Cellulose for use in the treatment according to any one of statements 33 to 40, wherein the average degree of polymerization of said cellulose is below 80.
42. Cellulose for use in the treatment according to any one of statements 33 to 41, wherein the average degree of polymerization of said cellulose is below 60.
43. Cellulose for use in the treatment according to any one of statements 33 to 42, wherein the average degree of polymerization of said cellulose is below 50.
44. Cellulose for use in the treatment according to any one of statements 33 to 43, wherein the average degree of polymerization of said cellulose is below 40.
45. Cellulose for use in the treatment according to any one of statements 33 to 44, wherein the average degree of polymerization of said cellulose is below 30. Medical uses all combinations of above-mentioned DP values and above mention CI values are explicitly disclosed and envisaged.
46. Cellulose for use in the treatment according any one of statements 33 to 45, wherein the crystallinity index of said cellulose is below 0.33 and the average degree of polymerization is below 50.

Further disclosed herein is a method of producing a microcrystalline cellulose, the method comprising the steps of:
a) providing a composition comprising at least 60% (w/w) cellulose, wherein the cellulose has an average Degree of Polymerization (DP) of between 20 and 100, b) mechanically treating said composition until the crystallinity index of said cellulose is below 0.5 and the degree of polymerization is below 100.

In step a, said composition may comprises at least 70, 80, or 90% (w/w) cellulose.

In step b) the composition may be treated until the crystallinity index of said cellulose is below 0.45, below 0.40 below 0.35 or below 0.33.

In step b) the composition may be treated until the average degree of polymerization is below 80, below 60, below 50 below 40 or below 30.

In step b) the composition may be treated until the crystallinity index of said cellulose is below 0.33 and the average degree of polymerization is below 50.

The composition in step a) may be obtained via a method of mechanically treating a composition comprising cellulose and a subsequent method of hydrolysing said mechanically treated composition.

### DETAILED DESCRIPTION

### FIGURE LEGENDS

FIG. 1: XRD refractogram with the 1002 peak and the Iam minimum to define the Crystallinity Index.

FIG. 2: Crystallinity Index of Avicel cellulose after a mechanical pre-treatment in a planetary ball mill (BM).

FIG. 3: Average DP of modified cellulose in function of the insoluble yield. Modifications consist of a ball mill treatment (open squares), an acid hydrolysis under variable process conditions (red dots), or the same acid hydrolysis procedures after a ball mill pre-treatment of 6 h at 500 rpm (black dots).

FIG. 4: Average DP of ball milled Avicel cellulose, in function of the treatment time.

FIG. 5: Prediction profiler of the response surface model (r2=0.95), which describes the dependency of average DP on different process parameters. The grey numbers indicate the process parameters of SCC/SAC29.

FIG. 6: Average DP for unmodified and modified celluloses.

FIG. 7: SEM pictures of Avicel, BM (ball milled for 260 min at 400 rpm), SCC29 and SAC29.

FIG. 8: D50 values for the modified celluloses, measured with a laser diffraction particle size analyser.

FIG. 9: Strong water retention capacity of unmodified and modified celluloses

FIG. 10: Cellulose conversion for modified and unmodified Avicel, during a 1 h digestion with Cellic CTec2 enzyme blend at 40° C. High enzymatic accessibility of the cellulose results in a high conversion degree.

FIG. 11: pH of a faecal slurry with cellulose additions during fermentation at 37° C. SCFA production, induced by cellulose fermentation, decreases the pH.

FIG. 12: Production of Acetate, Propionate and Butyrate during the in vitro fermentation.

FIG. 13: Production of Acetate, Propionate and Butyrate during the in vitro fermentation.

FIG. 14: pH of a faecal slurry with cellulose additions during fermentation at 37° C. SCFA production, induced by cellulose fermentation, decreases the pH.

FIG. 15: Detailed production of Acetate, Propionate and Butyrate during the in vitro fermentation

FIG. 16: pH of a faecal slurry with Avicel and inulin addition during fermentation at 37° C.

FIG. 17: Relative bread loaf volumes of breads of which 5, 10, 15 or 20% of the wheat flour is substituted by (modified) cellulose. Breads are made with water absorptions of 56.0, 58.2, 63.7 and 64.3% for 5, 10, 15 and 20% substituting levels respectively

FIG. 18: Relative bread loaf volumes of breads of which 20% of the wheat flour is substituted by (modified) cellulose. Breads are made with a water absorption of 64.3% (black) or maximal water absorption (grey) which is 76.3% for the bread with Avicel incorporation and 69.3% for the bread with S-SCC incorporation

FIG. 19: Crumb and crust evaluation of Breads with 20% Avicel, oven-dried SCC and SAC. All breads were baked at their optimal water absorption (76.3%, 64.3% and 64.3% respectively.

### DEFINITIONS

Avicel: Commercial cellulose preparation, with an average DP of around 160 AGU, and a crystallinity index (CI) of 0.72-0.87 (depending on resource).

BM: Cellulose, treated in a ball mill [treatment A]. These ball-milled celluloses have a CI under 0.33, and DP of 50-160 AGU (depending on the severity of the treatment).

SCC fibre: Cellulose after a consecutive ball mill and acid hydrolysis treatment [treatment A+B]. These fibres have a DP<100, and CI above 0.50.

SAC fibre: Cellulose after a consecutive ball mill, acid hydrolysis and another ball mill treatment [treatment A+B+C]. These fibres have a DP<100, and CI under 0.33.

### Process Optimization for SCC Fibres

A decrease in the average DP is obtained by acid hydrolysis. Acid hydrolysis preferably takes place in amorphous zones of cellulose, which can be incorporated randomly in the crystalline chains of Avicel cellulose by a ball mill pre-treatment [Zhao et al. (2012) Biofuels, Bioproducts and Biorefining 6 (4), 465-482; Lin et al. (2010) Applied biochemistry and biotechnology 162 (7), 1872-1880]. FIG. 2 shows the decrease in crystallinity index (CI) during a ball mill treatment at 500 rpm, in function of the treatment time. After a treatment of 120 minutes, the crystallinity index was 0.33, which is at the lower limit of the peak height method of Segal et al. If more amorphicity is induced in the fibres, the amorphous signal in the X-ray diffraction is overlaying the crystalline peaks, making the Iₐₘ valley invisible.

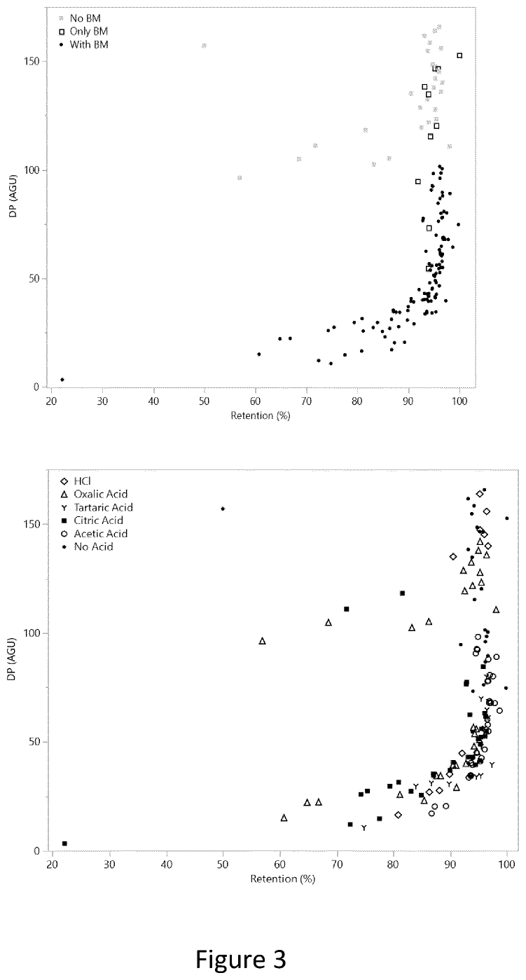

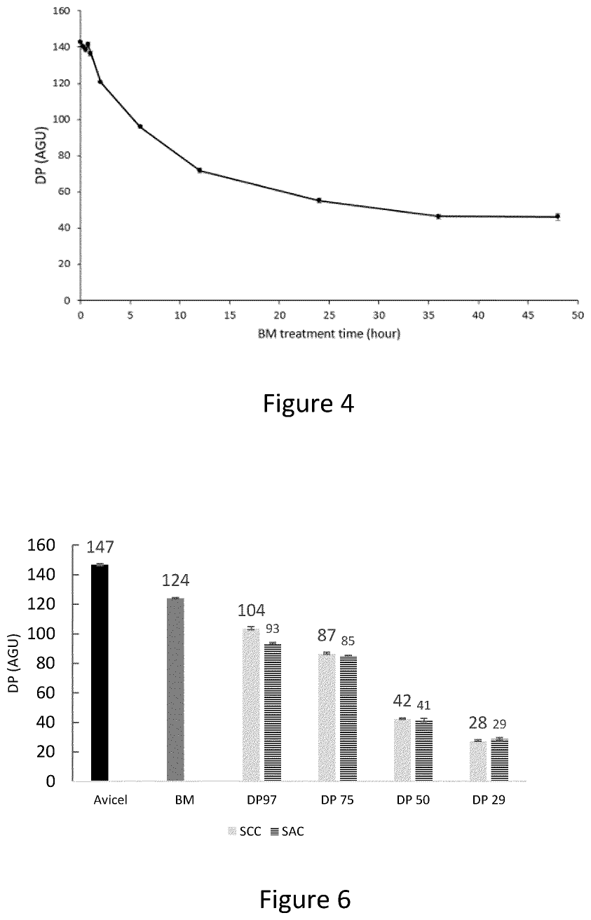

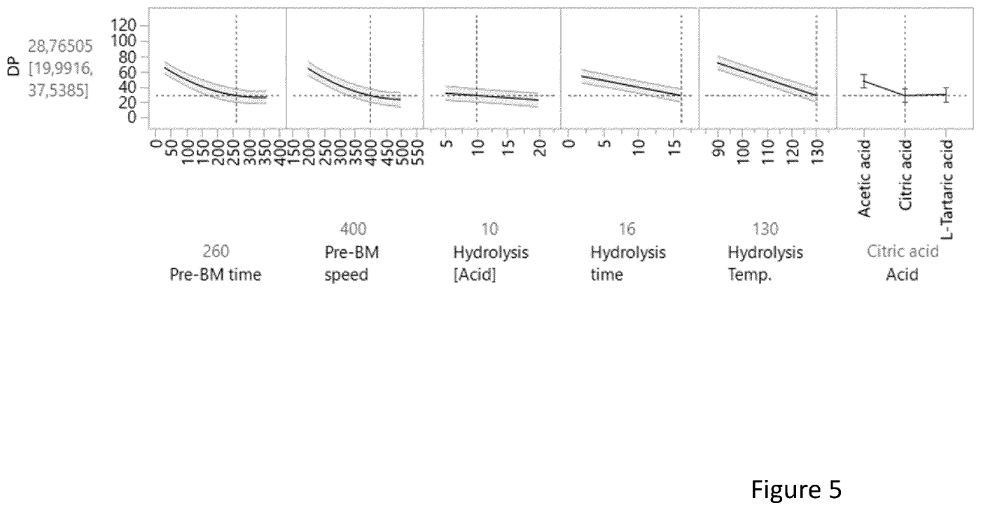

The effect of this decrease in crystallinity, prior to the acid hydrolysis is shown in FIG. 3. Herein, untreated Avicel (Average DP of 160 anhydroglucose units (AGU)) and ball milled Avicel (6 h at 500 rpm) was hydrolysed with HCl, oxalic acid, tartaric acid, citric acid or acetic acid under different processing conditions (acid concentrations 0.0005%-50%, hydrolysis temperatures 100° C.-120° C., hydrolysis time 0.5-16 h). The average DP of the insoluble fibres after modification is given in function of their insoluble yield. This insoluble yield is important, since part of the cellulose chains are lost through solubilization of the shortest fibres (DP 1-8) [Shrotri et al. (2013) Green Chemistry 15 (10), 2761-2768]. The open squares represent the cellulose fibres after just a ball mill treatment (0.25 h-48 h, 500 rpm), while the grey squares represent cellulose fibres after a hydrolysis procedure, without the prior ball mill pre-treatment.

For any type of acid, ball mill pre-treatment is necessary to obtain a high yield of SCC fibres with a DP, lower than 90 AGU. Inducing amorphous zones in the long fibres during the pre-treatment results in a lower Level-off degree of polymerization (LODP), which is the lowest average DP that can be reached, without a significant loss in insoluble material. This average DP is typically reached when the easily accessible (amorphous) zones are hydrolysed, and the acid starts to hydrolyse crystalline zones very slowly [Calvini (2005) Cellulose 12, 445-472].

A hydrolysis procedure without the ball mill pre-treatment is, hence, not suitable for the production of SCC fibres with a DP<50 AGU with yields above 90%, neither is a ball mill treatment on its own.

TABLE 2
Effect summary of the response surface model (r² = 0.95),
which describes the dependency of average DP
on different process parameters

| Input parameter | LogWorth | PValue |
|---|---|---|
| Hydrolysis Temp. | 13.826 | 0.00000 |
| Pre-BM time | 13.048 | 0.00000 |
| Pre-BM speed | 11.990 | 0.00000 |
| Hydrolysis time | 8.169 | 0.00000 |
| Acid | 5.752 | 0.00000 |
| Pre-BM time*Pre-BM time | 2.722 | 0.00190 |
| Pre-BM speed*Pre-BM speed | 2.364 | 0.00432 |
| Hydrolysis [Acid] | 2.056 | 0.00878 |

Table 2 shows that the temperature during hydrolysis is of importance for decreasing the average DP of the insoluble fibres. Also, the presence and intensity of the ball mill pre-treatment are of importance to ensure efficient hydrolysis towards low DP values. The weaker monocarboxylic acid, acetic acid (pka=4.75), is less efficient in decreasing the average DP of the cellulose in comparison to the dicarboxylic and tricarboxylic acids (L-Tartaric acid and Citric acid). A stronger acid such as citric acid or tartaric acid is typically used to shorten the processing time but does not influence the insoluble yield.

### Characterization of Avicel, BM, SCC and SAC Fibres

The starting material Avicel has a measured average degree of polymerization of 140-170 AGU [Deneyer et al. (2016) Green Chemistry 18 (20), 5594-5606]. This average DP is lowered through the modification in the first ball mill treatment towards 124AGU (FIG. 6). The combination of a BM pre-treatment and an acid hydrolysis with citric acid allows decreasing the average DP further towards 29AGU with an insoluble yield of >98%.

The further ball mill post-treatment has a limited effect on the average DP of the insoluble fibres obtained after hydrolysis. However, the ball mill post-treatment further decreases the crystallinity of the cellulose fibres, as shown in table 3.

A ball mill post-treatment of only 30 min allows to break down the crystallinity of SCC fibres, since the XRD analysis only could detect amorphous signal, resulting in a crystallinity index <0.33.

TABLE 3
Crystallinity index for Modified celluloses

| | Crystallinity index |
|---|---|
| Avicel | 0.72 |
| BM | <0.33 |
| SCC96 | 0.51 |
| SCC75 | 0.47 |
| SCC50 | 0.56 |
| SCC29 | 0.62 |
| SAC29-SAC50-SAC75-SAC96 | <0.33 |

TABLE 4
Overview of the variations on SCC and SAC, with their
corresponding treatment.

| | Avicel | BM | SCC | SAC |
|---|---|---|---|---|
| DP | ±160 | 124 | 29, 50, 75 or 97 | 29, 50, 75 or 97 |
| [AGU] | | | (SCC29, | (SAC29, |
| | | | SCC50, SCC75, | SAC50, SAC75, |
| | | | SCC97 | SAC97 |
| | | | respectively) | respectively) |
| CI | 0.70-0.90 | <0.33 | 0.50-0.80 | <0.33 |
| Treat- | None | Ball mill | Ball mill and Acid | Ball mill, Acid |
| ment | | 260 min | hydrolysis (10% | hydrolysis and |
| | | @ | Citric acid) | subsequent |
| | | 400 rpm | SCC29: 16 h, 130° C. | Ball mill |
| | | | SCC50: 16 h, 110° C. | subsequent |
| | | | SCC75: 2 h, 110° C. | Ball mill: |
| | | | SCC97: 2 h, 90° C. | 30 min @500 |
| | | | | rpm |

### Aggregate Structure

SAC fibres differ from Avicel, BM and SCC fibres due to a combination of both low average DP and low crystallinity. These fibres, however, are organized in larger aggregates, and the modification is also influencing the shape, size and properties of these aggregates.

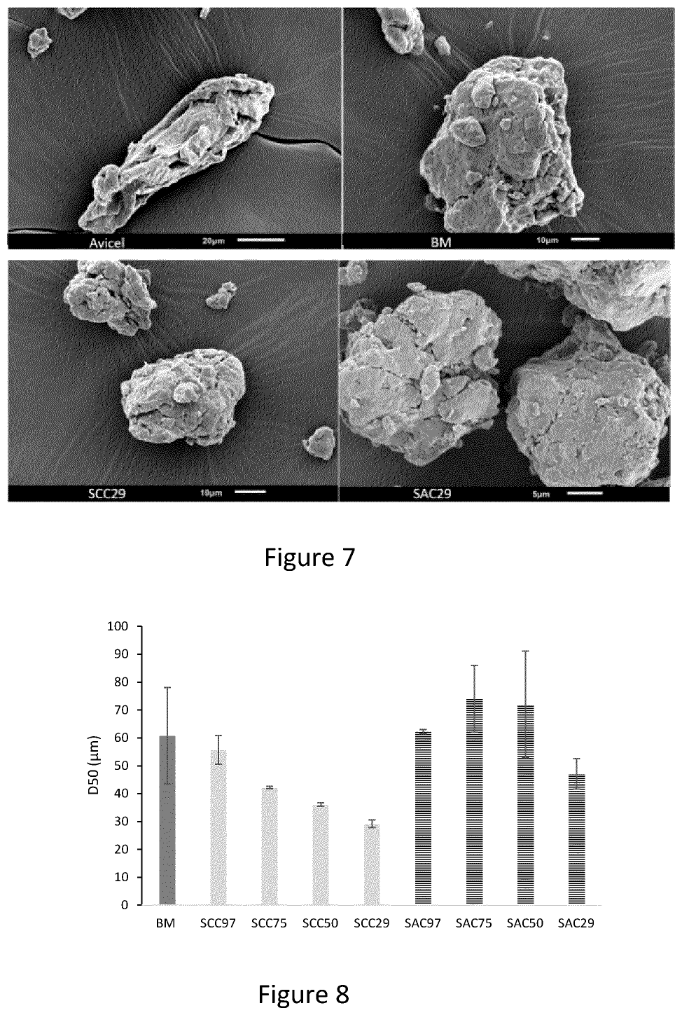

The aggregate shape of unmodified Avicel, ball milled Avicel, SCC29 and SAC29 are shown in FIG. 7. For the starting Avicel material, the cellulose fibres are organized in rod-like aggregates with an irregular surface. Dense fibre bundles are still visible on the surface of the aggregates, as well as large macropores between them. A ball mill treatment converts these rod-like aggregate shapes into spherical aggregates. Macropores at the surface disappear through the systematic disruption and re-aggregation of the cellulose fibres, and this disappearance already showed up after 15 minutes in the ball mill. However, further treatment time still causes further re-organization of the fibres within the spherical aggregates. The density of the fibres seems higher since the surface is denser, and no macropores are visible anymore. The average particle size is decreased by ball milling, through spheres with an average diameter of 60.7 μm (FIG. 8). Hydrolysis of the ball-milled cellulose had limited influence on the aggregate shape, neither on the appearance of the aggregate surface. However, the hydrolysis of the cellulose caused a further decrease in particle size, depending on the severity of the hydrolysis (FIG. 8).

When the SCC fibre suspension was dried in the spray dryer, smaller spherical particles, with an average particle size of only 13 μm.

The conversion of the SCC to the SAC is linked to an increase in particle size again, thanks to the compressions occurring in the ball mill. The average diameter is increased towards 47.1 μm for the SAC29, and this effect is even more pronounced for the longer SAC's. The shape of the aggregates is not influenced through this additional ball mill step: the dense spherical aggregates with regular surface stay visible on the SEM pictures.

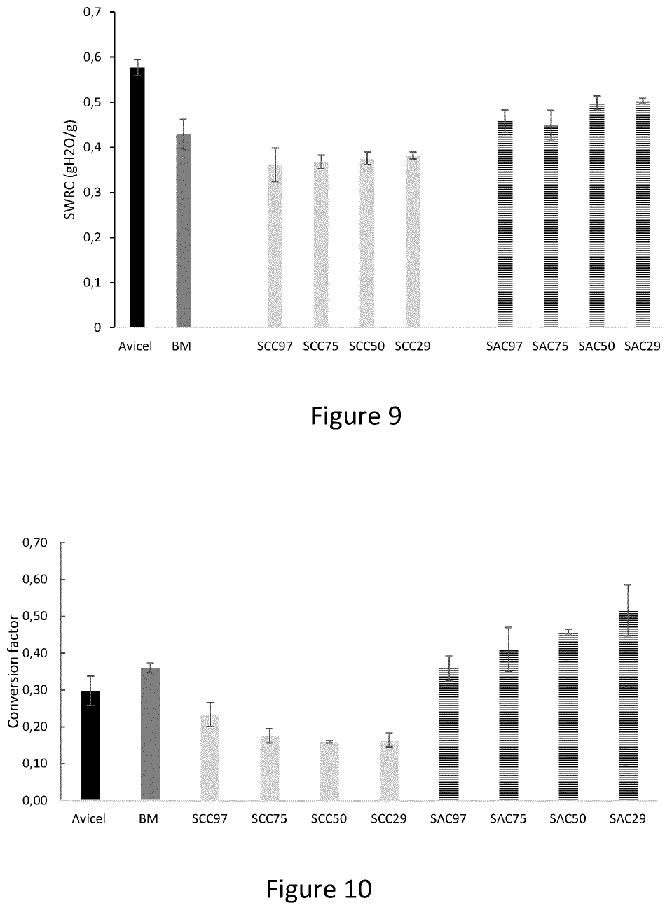

The changes in aggregate structure are translated into a different behaviour of water, when it is in contact with the celluloses. In FIG. 9, the strong water retention capacity (SWRC) of the celluloses is depicted.

The strong water retention capacity (SWRC) was determined in triplicate based on the method described by De Bondt et al. [De Bondt et al. (2020), Food chemistry, 305, 125436]. Cellulose (50 mg) was added in the upper part of a QIAprep Spin Miniprep Column (Qiagen) together with 700 μl deionised water. After 1 h hydration, the Miniprep was centrifuged for 10 min at 4000 g, and the upper part (with hydrated sample) was weighed (m_{centr}). The SWRC was calculated from Strong water retention capacity (mL/g)=(m_{centr}−m_{blanc}−m_{DM})/m_{DM}; wherein m_{blanc} represent the mass of the upper part after a blank measurement (without sample), and m_{DM} the mass of dry matter of cellulose in the column.

Unmodified Avicel has the highest SWRC (0.577 g H₂O/g±0.018) due to the presence of macropores wherein water is entrapped easily. Disruption of those microfibers and macropores causes a decrease in SWRC for the ball-milled sample. Hydrolysis of the ball-milled cellulose is causing a further decrease, which feeds the suggestion that also a measurable amount of water is bound within the amorphous regions of the cellulose, which disappear during the hydrolysis. Furthermore, the amorphous zones are returning in the conversion from SCC to SAC, which increases water binding again.

### Functionalities

### Digestibility/Fermentability

Due to the unique combination of the lower DP and crystallinity, enhanced overall accessibility can be expected for the SAC fibres, in comparison to prior art microcrystalline cellulose [Liao et al. (2020) Molecular Catalysis 487, 110883]. In FIG. 10, the cellulose conversion after a 1 h digestibility experiment with a CTec2 cellulase blend is shown. High accessibility for the cellulases will result in a high conversion factor, while the most recalcitrant fibres will have the lowest conversion factor. For unmodified Avicel cellulose, 30.0%±4.0 of the cellulose was converted to glucose and cellobiose after 1 h digestion, and a ball mill treatment was already increasing this conversion factor. The SCC fibres are all less digested than unmodified or ball milled Avicel, showing that a decrease in average DP is not sufficient for improving the enzymatical accessibility of cellulose. The compaction during the ball mill and the removal of amorphous zones during the acid hydrolysis apparently have more effect on the digestibility than the DP decrease, which is expected to influence the digestibility positively. However, if amorphous zones are incorporated in the SCC fibres again (SAC fibres) through the ball mill post-treatment, it is clear that the enzymatic accessibility is exceeding the values of unmodified Avicel or BM cellulose. Depending on the average DP, the digestibility with the CTec2 enzyme blend can be increased to 52.6%±6.9. Therefore, the combination of both low DP and low crystallinity allows enhancing the accessibility of the cellulose.

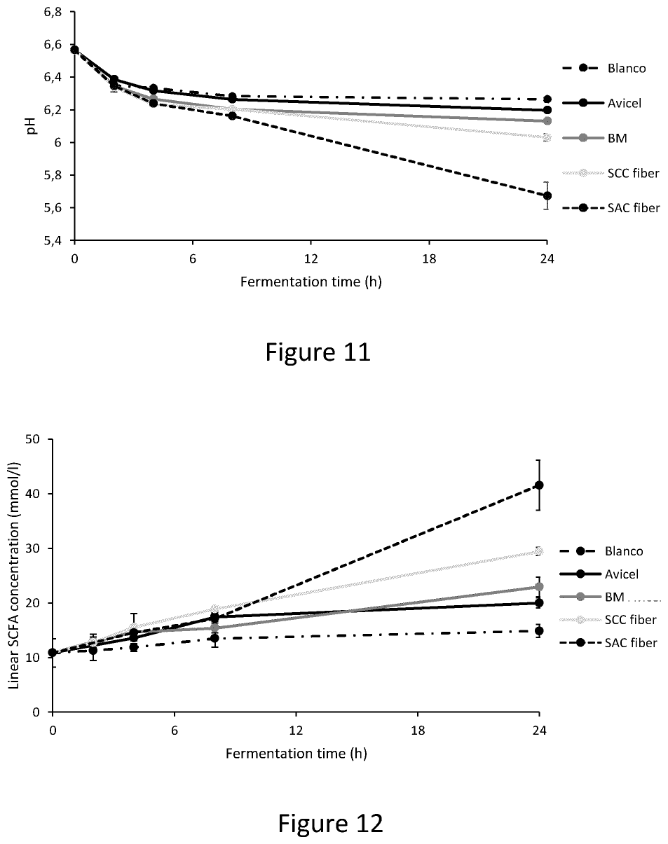

To translate this enhanced enzymatic accessibility into food functionality, the behaviour of the celluloses in the human large intestine is evaluated with an in vitro approach. The pH during fermentation at 37° C. of a faecal slurry with the addition of untreated, ball-milled, SCC or SAC fibres from Avicel is shown in FIG. 11. SCFA production, induced by carbohydrate fermentation, will decrease the pH.

The fermentability of cellulose after 24 h is increased when the Avicel is ball milled once or ball-milled and subsequently hydrolysed with citric acid. This shows that decreasing the crystallinity or decreasing the average DP of cellulose is improving the accessibility of cellulose for the gut microbiota. The largest pH drop is obtained when the SAC fibres were added. Also in this in vitro trial, decreasing both the DP and the crystallinity significantly decreases the pH of the lumen. This pH decrease is positive for human health since pathogen growth is repressed and proteolytic fermentation, which produces several toxic metabolites, is inhibited [den Besten et al. (2013) J. lipid research 54, 2325-2340].

The production of SCFA during this in vitro fermentation experiment is determined with gas chromatography. Acetate, butyrate and propionate are the main SCFA from carbohydrate fermentation in the colon and are named ‘linear SCFA’. FIG. 12 shows that more linear SCFA were produced in the faecal slurry upon the addition of SAC fibres. The final concentration of acetate, propionate and butyrate in the faecal slurry with SAC fibres was 41.54 mmol/l, while the control faecal slurry had only 14.85 mmol/l linear SCFA. The minimal degree of fermentability (MDOF) of the SAC fibres can be calculated based on the difference between the linear SCFA mass in the control faecal slurry and in the faecal slurry with SAC addition. At least 45.8% of the mass of the SAC fibres was fermented into acetate, propionate and butyrate after 24 h, while the MDOF of untreated Avicel and BM Avicel was only 7.6% and 13.3%, respectively. The actual fermentation degree will always be even higher than the MDOF since part of the mass will also be converted to lactate, water, H₂, . . . which are not analysed in this experiment [Glenn et al. (1995) J. Nutrition 125 (6), 1401-1412]. SCC fibres were also able to increase the SCFA production (MDOF=30.8%), but the further mechanical treatment after the hydrolysis results in the highest fermentation degree. Next to the enhanced production of SCFA, there was an enrichment in Bifidobacteria detected after the fermentation experiment, in the faecal inoculum that was enriched with the SAC fibres.

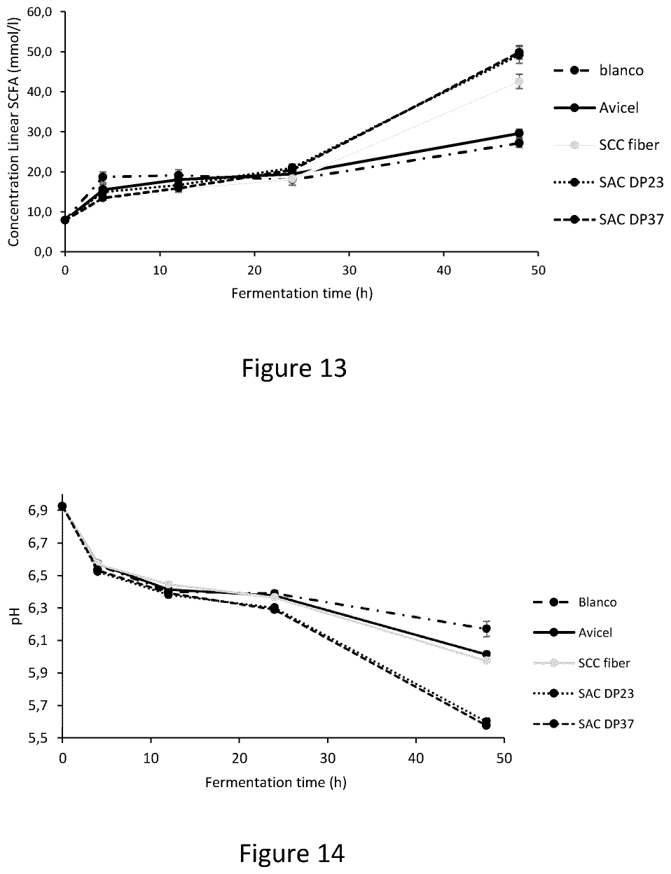

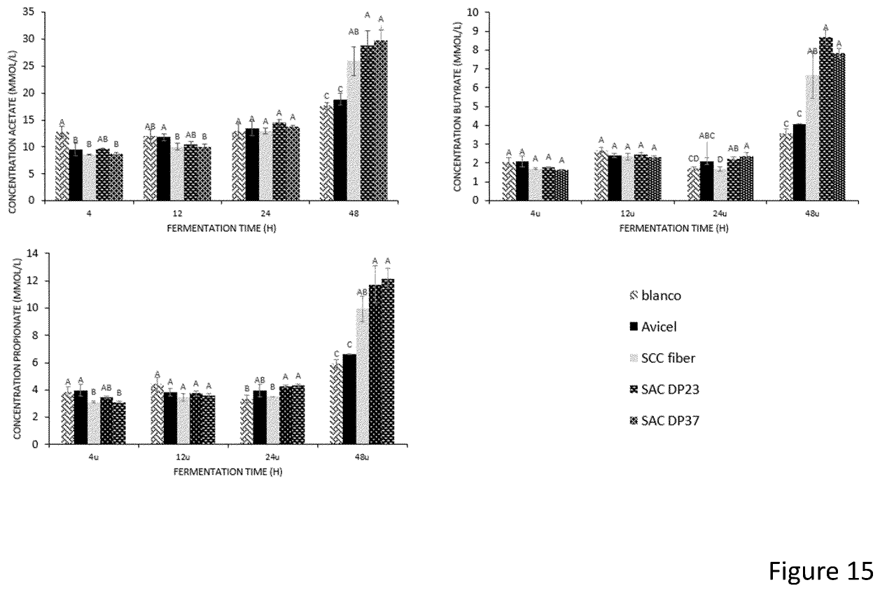

In a second in vitro fermentation trial, the influence of DP within the low DP ranges was investigated, comparing a SAC fibre with an average DP of 23 AGU (SAC DP23) with a SAC fibre with an average DP of 37 (SAC DP37) (FIG. 13). Contrary to previous experiments, the ball mill post-treatment was performed for 1 h instead of 30 minutes. The pH plot illustrates that the cellulose fibres are mainly fermented between 24 and 48 h, while in the previous experiment, the largest pH drop was obtained between 8 and 24 h. This difference is likely due to the use of the faeces of different donors. However, also here, SAC fibres are better fermentable than the untreated Avicel or SCC fibre.

Also, in the production of linear SCFA, the same effect can be detected: The SCFA production follows the same kinetics as the pH progress, and the DP difference between 23AGU and 37AGU is not sufficient to make significant differences in SCFA production during an in vitro fermentation trial. The addition of untreated Avicel did not significantly increase the SCFA content in the faecal slurry, which means that the fermentation of the Avicel is negligible again (MDOF of 5.5%). However, the addition of the SAC fibres could increase the content of linear SCFA in the faecal slurry, resulting in an MDOF 46% for both SAC fibres. Despite the individual variability of the faeces donors, the degree of fermentation is comparable with the previous in vitro fermentation experiment.

Microbial analyses, however, demonstrated that this fermentation was mainly driven by Ruminococcus species.

Next to the absolute production of acetate, propionate and butyrate, the relative production of this linear SCFA compared to the branched SCFA is investigated. These branched SCFA (isovalerate and isobutyrate) are products of protein fermentation, which are to be avoided as they may be further metabolized in the human body into toxic metabolites (e.g., phenolic compounds, ammonia, amines, . . . ) [Hughes et al., (2000) Current issues in intestinal microbiology 1, 51-58]. In Table 4, the ratio of branched SCFA from protein fermentation (isovalerate and isobutyrate) to total SCFA is shown for the faecal slurry after 48 h of fermentation. The addition of SAC fibres decreases this proportion from 8.45% to 6.01% or 6.04%, demonstrating that these fibres inhibit protein fermentation, which has a positive effect on colon health. The incorporation of the SCC fibre also resulted in a decreased relative protein fermentation as well, but the greatest effect is obtained if Short amorphous cellulose is incorporated.

TABLE 5
ratio of isovalerate and isobutyrate versus total SCFA

| Cellulose sample | Branched SCFA/Total SCFA |
|---|---|
| blanco | 8.45 |
| Avicel | 7.88 |
| SCC Fibre | 6.55 |
| SAC DP 23 | 6.01 |
| SAC DP 37 | 6.04 |

The different linear SCFA all have a distinct metabolic function in the human body. The physiologic effects of the SCFA furthermore depend on the relative amounts of acetate, propionate and butyrate [Wong et al. (2006) J. Clinical Gastroenterology 40 (3), 235-243]. The production of these SCFA is shown in FIG. 14. Wong et al. stated that the average proportion acetate:propionate:butyrate is 3:1:1 on average, which is also clear from the figure. However, the butyrate production is for these faecal slurries always lower than the propionate production. In the first phase of the experiment (0-24 h), mainly the acetate production is high, while the butyrate and propionate production are rather limited. Between 24 and 48 h, acetate is produced, together with higher amounts of butyrate and propionate. Propionate and butyrate are produced from secondary fermentation and consequently depend on the acetate content in the faecal slurry, which explains this behaviour [Wong et al., cited above]. When the ratio butyrate/linear SCFA is calculated, it is clear that the addition of SAC fibres also had an impact on the relative amounts of butyrate compared to the acetate production. Moreover, when adding SAC DP23 or SAC DP37, the ratio butyrate/linear SCFA is equal to 17.6% and 15.7%, respectively, while for the control faecal slurry, this ratio is only 13.1%. Enhanced production of butyrate is correlated to increased colon health and lower risk on inflammation, as the butyrate is the main energy source for the gut cells. Next to the decrease in (lumen) pH, inhabitation of the protein fermentation and enhanced production of SCFA, adding the SAC fibres to a faecal slurry also causes an increase in the relative amount of butyrate.

### Bread Incorporation

The SAC has a high micro-accessibility, resulting in the enhanced fermentability. On the other hand, the lowered strong water retention capacity demonstrates that the macro-accessibility of the SCC and SAC fibres is decreased. This causes additional techno-functional opportunities, when the modified cellulose is added to a bread dough recipe. FIG. 17 shows bread loaf volumes of breads with cellulose incorporation levels of 5, 10, 15 and 20%. The WA absorption for each incorporation level was 56.0, 58.2, 63.7 and 64.3% respectively. Incorporation of cellulose results in loaf volume loss, since the cellulose is dilution the gluten in the dough, which are necessary for the development of a visco-elastic network in the dough. Furthermore, they will hinder the gluten network formation sterically, and will compete with the gluten for water [Bock et al. (2013) Food Hydrocolloids, 31 (2), 146-155; Hemdane et al. (2016) Comprehensive reviews in food science and food safety, 15 (1), 28-42]. Increasing the cellulose content from 5% towards 10% is causing a first loaf volume reduction, while this reduction stays limited with further increase towards 15% incorporation. Between 15% and 20% incorporation, the additional loaf volume decrease depends on the aggregational properties of the cellulose. Avicel with its high SWRC faces an additional volume drop, while the loaf volume difference between 15% and 20% incorporation level stays limited for the Oven dried SCC (O-SCC) and SAC fibres. At 20% substitution level, the loaf volume reduction of a bread with Avicel is 65.6±1.4% compared to a control bread, while this is only 16.2±1.9% and 20.9±0.1% for O-SCC and SAC incorporation respectively. Gluten dilution and steric hindrance of the fibres can explain parts of this differences in volume losses, but dough handling also revealed that the water distribution is not optimal in the different doughs. Since Avicel (and Spray dried SCC) have the highest SWRC, an improper gluten hydration can be expected in the doughs with these enrichments. Therefore, adding more water to the dough recipes might reduce the detrimental effects of the cellulose addition, resulting in higher bread loaf volumes.

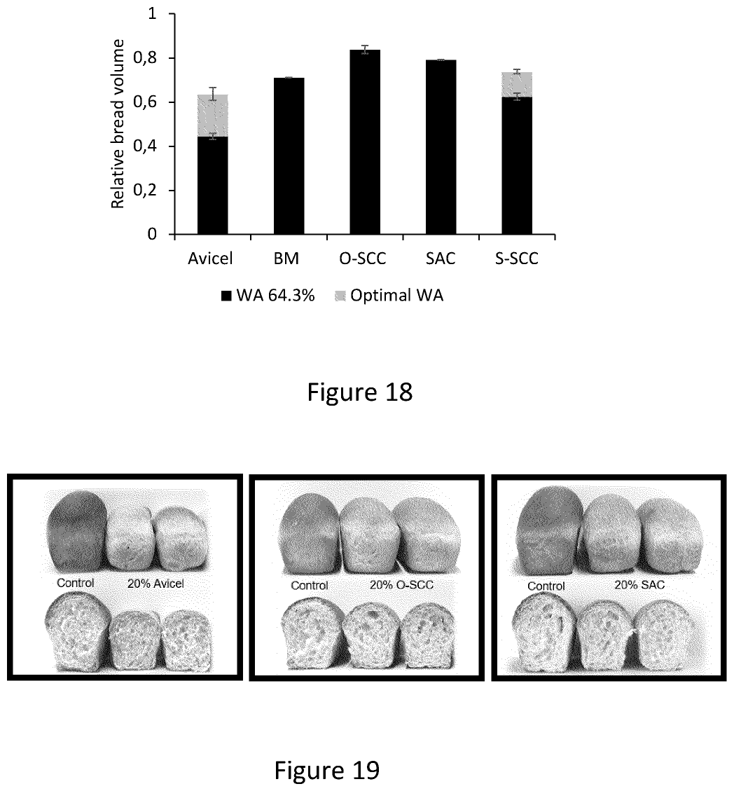

In FIG. 18, the relative bread loaf volumes of breads baked on standard (64.3%) and optimal WA with 20% cellulose incorporation are shown. The bread loaf volumes of breads with Avicel and Spray dried SCC fibres (S-SCC) could indeed be increased in this way. A dough with 20% Avicel could handle a WA of 76.3%, while the dough with 20% Spray dried SCC (S-SCC) could handle a WA of 69.3%. The maximal WA of O-SCC and SAC incorporated breads was slightly higher than 64.3% (66.3%), but these higher WA did not result in higher loaf volumes. Even with an optimal hydration level, the incorporation of 20% Avicel prevents a proper dough development, resulting in a loaf volume loss of 36.4±2.9%. The same holds for the S-SCC incorporation: the final loaf volume upon optimal hydration was still lower than for breads with BM, O-SCC or SAC addition. The lowered SWRC, (bio) chemical inertness and spherical shape of these modified celluloses makes them suitable for fibre enrichment in bread upon substitution levels of 20%.

In FIG. 19, the effect of 20% cellulose incorporation (Avicel, O-SCC and SAC) on crumb and crust colour of breads with optimal WA is shown. Next to volume loss, it is clear that the incorporation of all purified celluloses makes the crust colour lighter, while the crumb colour remains unchanged. Avicel incorporation has the biggest effect on crust colour, possibly caused by the higher amount of water that is present in the recipe.

### Fermentation of Inulin

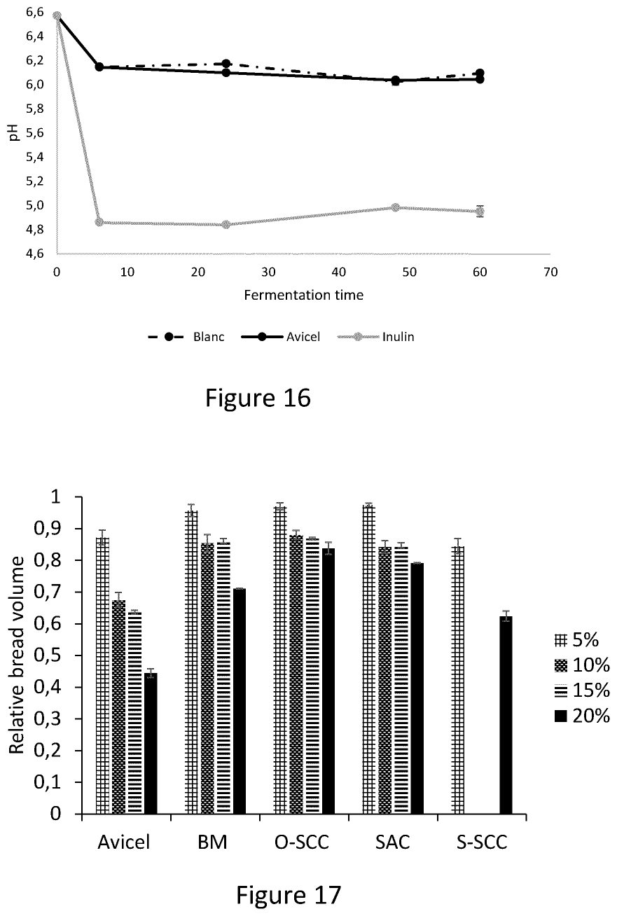

In FIG. 16, the pH in function of fermentation time is given for a faecal slurry enriched with Avicel or Orafti-HPX Inulin, in another independent in vitro fermentation trial. While SAC fibres are mostly fermented after a fermentation time of 8 h, Inulin is mainly fermented within the first two hours of fermentation. Furthermore, this Inulin fermentation causes a decrease in the pH of the faecal slurry, towards a value of 4.8, which is lower than the pH of SAC enriched faecal inocula in previous trials. Therefore, there can be stated that Inulin is fermented faster and to a higher extent then the SAC fibres. Consequently, for specific people with an increased sensitivity towards (fast) carbohydrate fermentation in the colon, the SAC fibres have additional advantages in terms of slowly providing SCFA to the colon microbiota without discomfort for the human host.

### Example 1 Process Description/Optimization

Avicel PH-101 (Sigma-Aldrich, Deurne, Belgium) [DP about 160 AGU, and a crystallinity index (CI) between 0.72 and 0.87] was modified towards short crystalline cellulose fibres (SCC fibres), by a subsequent combination of a treatment in the planetary ball mill pre-treatment (PM100, Retch GmbH) and an acid hydrolysis with mild organic acids (Sigma-Aldrich, Deurne, Belgium). After washing and drying, the SCC fibres were treated in the ball mill another time to produce short amorphous cellulose (SAC) fibres, which were expected to be more accessible for enzymatical or chemical reactions. To optimize and understand the modification process, different process parameters were varied, and the end products were evaluated on their average degree of polymerization (DP), crystallinity, and process yield. Afterwards, a selection of 10 samples was made for further analysis and valorisation examples. Both ball mill treatments (batches of 20 g) were conducted in a ball mill sample holder with a Zirconium oxide coating. Milling conditions prior to the hydrolysis procedure (referred to as “ball mill pre-treatment”) were varied in time and speed between 30 m and 6 h, and 200 rpm-500 rpm, respectively. On the contrary, the ball mill treatment after hydrolysis (“ball mill post-treatment”) was always performed at 500 rpm (0.5 h-1 h).

Hydrolysis was performed in an acid in water solution, with a solid to liquid ratio of 4.8%. Therefore, mild organic acids (hydrochloric acid (HCl), Oxalic acid (OA), Tartaric acid (TA), Citric acid (CA) and Acetic acid (AA)) were used in concentrations that varied between 2.5% and 50%, and hydrochloric acid was used in a concentration of 0.0005%. The temperature during these hydrolyses was varied between 90° C. and 130° C., while the stirring rate was always fixed at 800 rpm.

After hydrolysis, the insoluble material was washed with water until neutral pH, and dried in an oven for 48 h at 60° C., before undergoing the ball mill post-treatment. Alternatively, the SCC fibre suspension was dried in a mini spray drier B290 (BÜCHI Labortechnik GmbH, Hendrik-Ido-Ambacht, NL), or freeze-dried, after a snap freezing step with liquid nitrogen, or spray-dried before being treated in the ball mill. For the first screening experiments, hydrolysis was conducted in cellulose batches of 0.3 g, while the optimization of the production process parameters was performed with 15 g batches. For the process optimization, an I-optimal full factorial experimental design with response surface methodology was employed, using the JMP package. The six-factorial-three-level design with Ball mill speed, Ball mill time, Hydrolysis temperature, Hydrolysis time, Acid concentration and type of acid as input parameters required 42 experiments.

After optimization, a sample set of 10 celluloses (unmodified Avicel, ball milled Avicel, 4 SCC fibres and 4 SAC fibres) was selected for further characterization and evaluation of the functionalities. The ball mill pre-treatment was performed for 260 minutes at 400 rpm, while the hydrolysis was always performed with a 10% citric acid solution, with varying hydrolysis time and temperature (Table 1). The distinction between the SAC and SCC fibres consists of the absence or presence of a BM post-treatment (30 m at 500 rpm) or not.

TABLE 1
Hydrolysis conditions of the selected SCC and SAC fibres

| | Hydrolysis | Hydrolysis |
|---|---|---|
| | time (h) | Temperature (° C.) |
| SCC29/SAC29 | 16 | 130 |
| SCC50/SAC50 | 16 | 110 |
| SCC75/SAC75 | 2 | 110 |
| SCC97/SAC97 | 2 | 90 |

### Example 2 Characterization of Cellulose

De average degree of polymerization (DPᵥ) of the cellulose is defined as the average length (expressed in anhydroglucose units (AGU)) of the glucose polymers in a cellulose fiber. This DPᵥ is determined viscometrically with a capillary viscometer, based on an NF G 06-037 norm of the French institute for normalization (AFNOR). Purified cellulose samples (0.075 g) are dissolved in a 0.5M Bis (ethylenediamine) cupper (II) hydroxide solution (15 ml), and the viscosity of this solution at 25° C. is measured with a Schott Gerä the capillary viscometer, type nr. 509 04. The DPᵥ, expressed in anhydroglucose units [AGU], is calculated from the boundary viscosity of the solution (n), based on an empirical relation: DPᵥ^{α}=η/K, where α and K are empirical constants, equal to 1 and 7.5·10⁻³ respectively. The boundary viscosity η is determined from ηₐ=η·C·10^{(0.14·η·C)}, where ηₐ is the specific viscosity of the solution, and C is the concentration of cellulose per 100 ml.

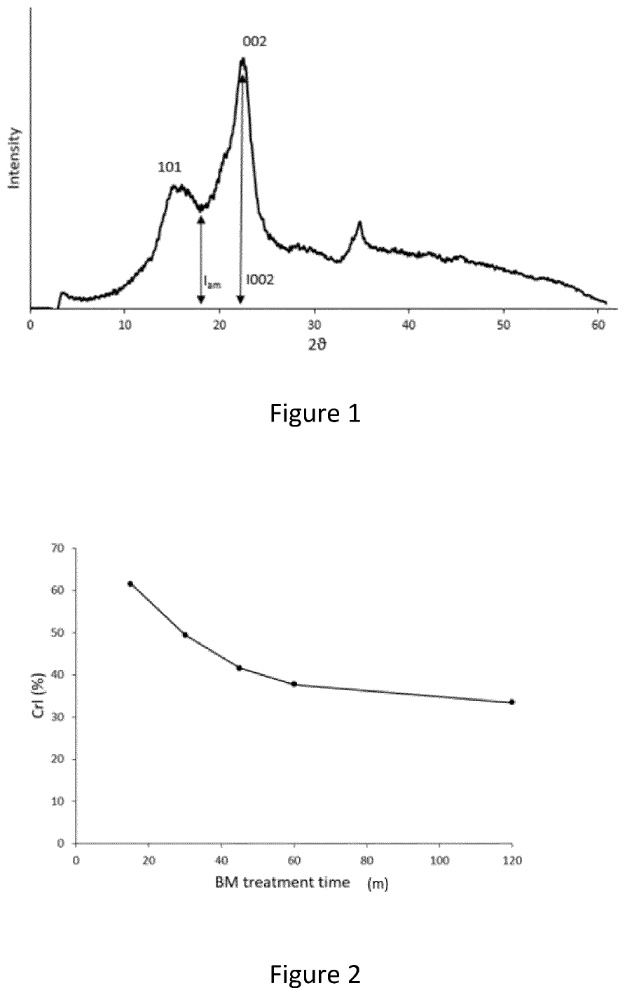

The crystallinity index (CI) is determined with x-ray diffraction (XRD) measurements based on the peak height method developed by Sega et al. (1959) Textile Res. J. 29 (10), 786-94]. The CI is calculated from the ratio of the height of the 002 peak (1002) and the height of the minimum between the 1002 and the 101 peaks (Iₐₘ) around 18°, as shown in FIG. 1. When a crystallinity index of 0.33 is reached, the Iₐₘ-minimum is not visible anymore. Amorphous samples of which the Iₐₘ-minimum is not possible anymore, are getting a CI of <0.33.

The aggregate size and aggregate shape influence the accessibility of the cellulose since the reactive surface largely depends on these characteristics [Gusakov (2007) Biotechnology and Bioengineering 97 (5), 1028-1103]. The aggregate size of the cellulose is determined with an LS 13320 laser diffraction particle size analyser (Beckman Coulter). A He-Ne laser beam is emitted on a solution of the cellulose in water, and the volumetric PSDs are calculated from the intensity profile of the scattered light with the Mie theory using the instrument's software. Furthermore, the agglomerates are visualized with a scanning electron microscope (SEM). A JEOL JSM-6010 JV microscope is used after coating the cellulose with a JEOL JSC-1300 sputter. The enzymatic digestibility is determined by calculating the enzymatic conversion (EC) after reaction with Cellic CTec2 cellulase, as described by Chen et al. (2015) Applied Energy 150, 224-232]. Cellulose is suspended (1% w/v) in a 50 mM Sodium acetate buffer (pH 4.8) and stirred at 900 rpm together with 20U/g cellulose Cellic CTec2 cellulase (Sigma-Aldrich, Deurne, Belgium). After 1 hour of incubation at 40° C., the enzymes are denatured by heating the solution (5 min@110° C.) and the solid fraction is separated from the supernatant by centrifuging at 5000G. The amount of glucose and cellobiose in the supernatant, which is formed from cellulose hydrolysis, is determined by High-performance-anion-exchange chromatography with pulsed amperometric detection (HPAEC-PAD) on a Dionex ICS3000 chromatography system (Sunnyvale, CA, USA). Saccharides were separated on a CarboPac PA-100 column (4×250 mm), equilibrated with 90 mM NaOH, by applying a sodium acetate gradient. The enzymatic conversion (%) is calculated from the amount of glucose (m_{g}) and cellobiose (m_{cb}) in the supernatant, and the amount of starting substrate (m_{c}):

$$EC ( \% ) = \frac{{m}_{g} + {m}_{cb}}{1.1 {m}_{c}}$$

### Example 3. In Vitro Fermentation Tests

In vitro fermentation tests were performed, following the procedure of De Preter et al (2010) Molecular nutrition & food research 54 (12), 1791-1801. In this set-up, 100 mg of the cellulose fibres are added to 25 ml of a 10 wt % faecal slurry, consisting of human faeces from 8 donors in phosphate-buffered saline (pH 7.3). The tubes are incubated anaerobically at 37° C. for 48 h, under continuous shaking. During this incubation, the colon microbiota from the faeces ferments the (accessible) carbohydrate fibres under ideal circumstances, resulting in the production of short-chain fatty acids. After incubation, the pH of the slurry is measured with a Hanna Instruments HI 9025 digital pH meter, and the production of short-chain fatty acids (SCFA) is quantified.

Only the amounts of acetate, propionate, butyrate, isobutyrate and isovaleric acid in the faecal slurry are determined with the gas-chromatographic method of Van de Wiele et al. (2007) J. Applied Microbiology 102 (2), 452-460. In this procedure, salts of the SCFA are extracted in diethyl ether. To create and neutralize the salts of the fatty acids, a 25% (w/v) NaOH and 50% sulfuric acid solution is added to the faecal slurry before extracting the SCFA from the faecal slurry to the diethyl ether phase. An Agilent 6890 Series gas chromatograph is used with an EC-1000 Econo-Cap column (25 m×0.53 mm, 130° C., 1.2 μm film thickness) with helium (20 ml/min) as carrier gas. A flame ionization detector at 195° C. measured the different fatty acids.

### Example 4. Preparation of Bread

Bread was made according to the straight dough procedure of Shogren and Finney (Shogren & Finney, 1984). In a 10 g pin bowl mixer (National Manufacturing, Lincoln, NE, USA), 10.0 g of flour-cellulose mixture (14% moisture content) was mixed at 23° C. for 240 seconds with 5.3% compressed fresh yeast, 6.0% sucrose, 1.5% salt and water. The water absorption (56%-76%) and cellulose content (5%-20%) were varied. The mixed doughs were fermented for 90 minutes in a fermentation cabinet (National Manufacturing) at 30° C. and relative humidity of 90%. Punching steps were performed with a dough sheeter (National Manufacturing) after 52, 77 and 90 min of fermentation. After the last punching, the dough was moulded and proofed for another 36 min in a slightly greased baking tin (25 mm×25 mm×50 mm). Doughs were baked for 13 min at 232° C. in a rotary oven (National Manufacturing). After 30 min cooling, the bread loaf volume was determined with a Volscan Profiler (Stable Micro Systems, UK). Bread volumes are always shown relatively to a control bread without cellulose addition.

1-43. (canceled)

44. A bakery product comprising a cellulose, the cellulose having:
an average degree of polymerization less than 100, as determined viscometrically; and
a crystallinity index less than 0.5, as determined by x-ray diffraction.

45. The bakery product according to claim 44, wherein the bakery product is a bread.

46. The bakery product according to claim 44, wherein the bakery product is a wholemeal bread.

47. The bakery product according to claim 44, comprising from 5% to 30% by weight of the cellulose, based on the total weight of the bakery product.

48. The bakery product according to claim 44, wherein the cellulose has a fermentation degree from 7.6% to 45.8%.

49. The bakery product according to claim 44, wherein the crystallinity index of the cellulose is less than 0.45.

50. The bakery product according to claim 44, wherein the average degree of polymerization of the cellulose is less than 80.

51. The bakery product according to claim 44, wherein the average degree of polymerization of the cellulose is less than 50 and the crystallinity index of the cellulose is less than 0.33.

52. A method of treating an inflammation from a colon disorder in a subject, the method comprising:
administering a cellulose to the subject, the cellulose having:
an average degree of polymerization less than 100, as determined viscometrically; and
a crystallinity index less than 0.75, as determined by x-ray diffraction.

53. The method according to claim 52, wherein the average degree of polymerization of the cellulose is less than 100 and the crystallinity index of the cellulose is less than 0.5.

54. The method of claim 53, wherein the cellulose is in a bakery product.

55. The method of claim 54, wherein the bakery product is a bread.

56. The method of claim 54, wherein the bakery product is a wholemeal bread.

57. The method of claim 54, wherein the bakery product comprises from 5% to 30% by weight of the cellulose, based on the total weight of the bakery product.

58. The method according to claim 52, wherein the cellulose has a fermentation degree from 7.6% to 45.8%.

59. The method according to claim 52, wherein the crystallinity index of the cellulose is less than 0.45.

60. The method according to claim 52, wherein the average degree of polymerization of the cellulose is less than 80.

61. The method according to claim 52, wherein the average degree of polymerization of the cellulose is less than 50 and the crystallinity index of the cellulose is less than 0.33.
